(19) United States
(12) Plant Patent Application Publication
(10) Pub. No.: US 2003/0150032 P1
(43) Pub. Date: Aug. 7, 2003

(54) Lamium maculatum 'Purple Dragon'

(76) Inventor: John vanSanden, Whitehall, MI (US)

Correspondence Address: David S. MacKenzie, Hortech, Inc., P.O. Box 533, Spring Lake, MI, 49456, US

(21) Appl. No.: 10/061,483

(22) Filed: Feb. 4, 2002

(51) Int. Cl. A01H 5/00

(52) U.S. Cl. PLT/263

(57) ABSTRACT

A spotted deadnettle variety named ‘Purple Dragon’, characterized by silver and green variegated leaves and unique in having rich purple colored flowers.

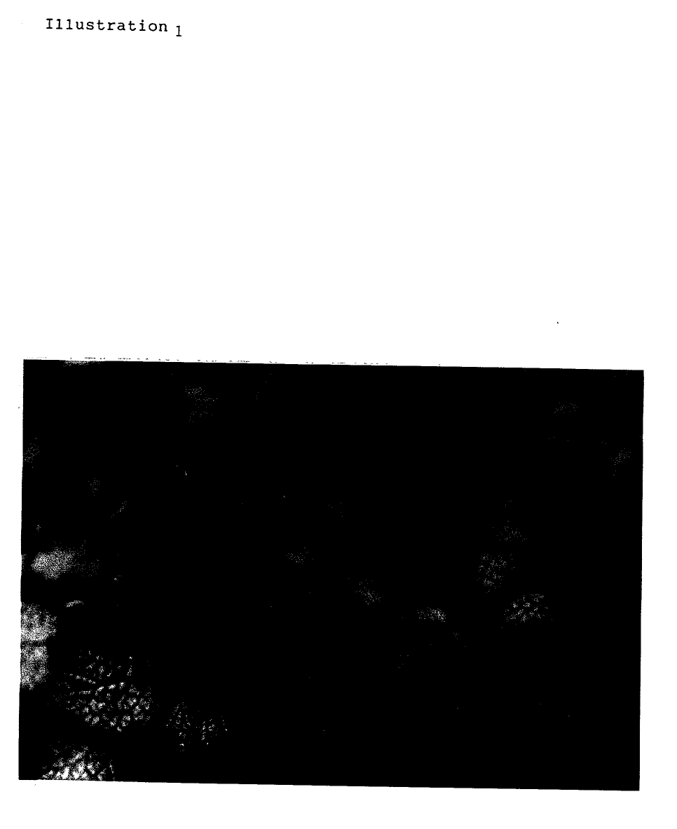

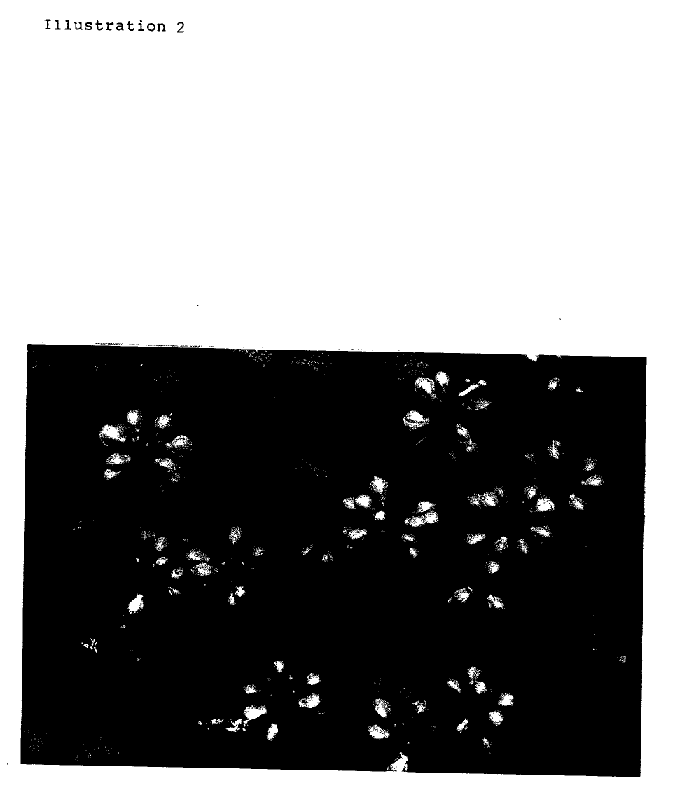

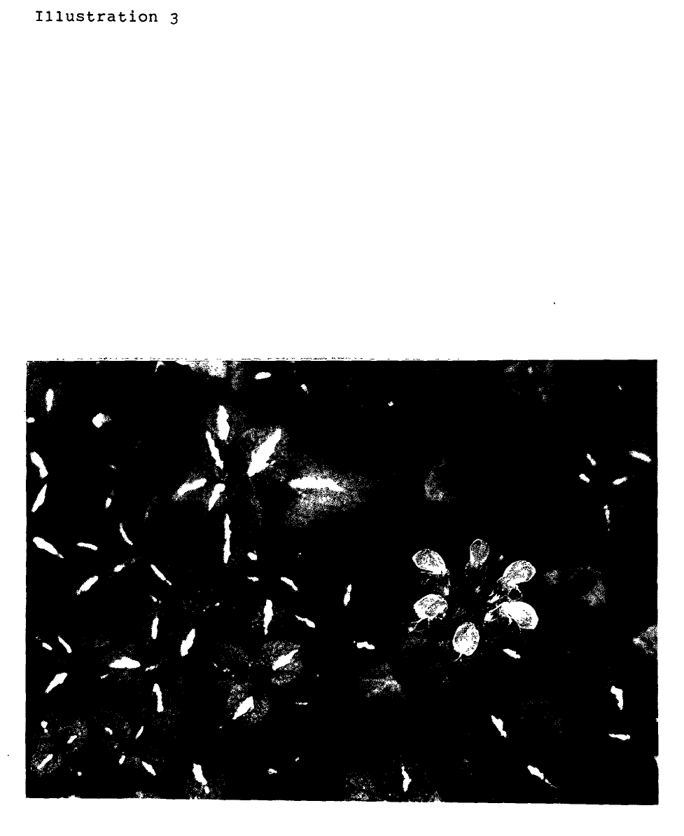

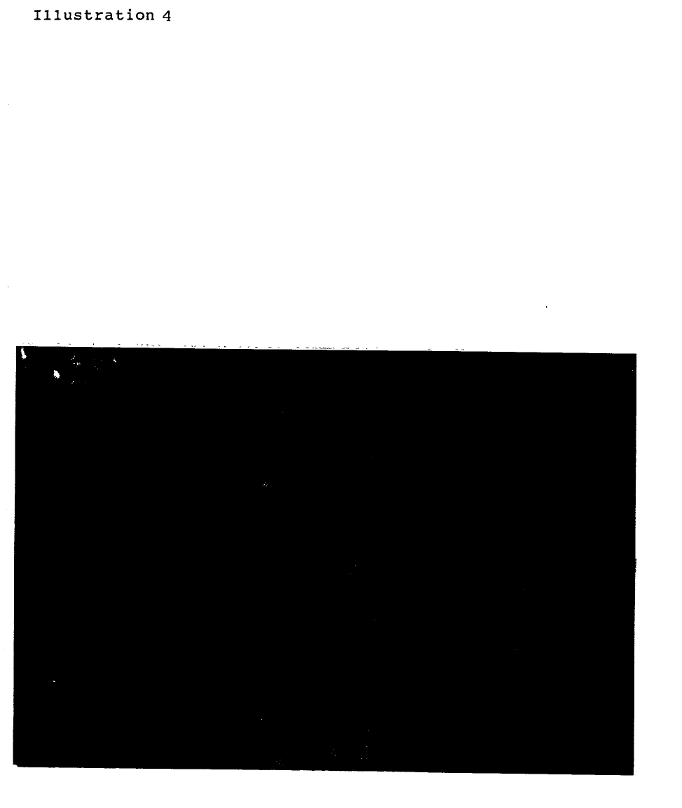

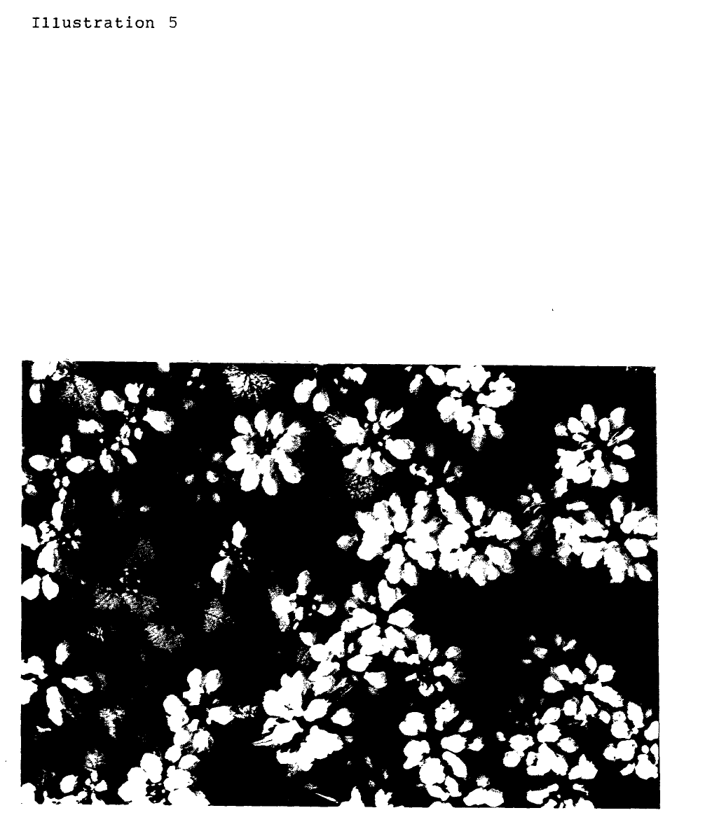

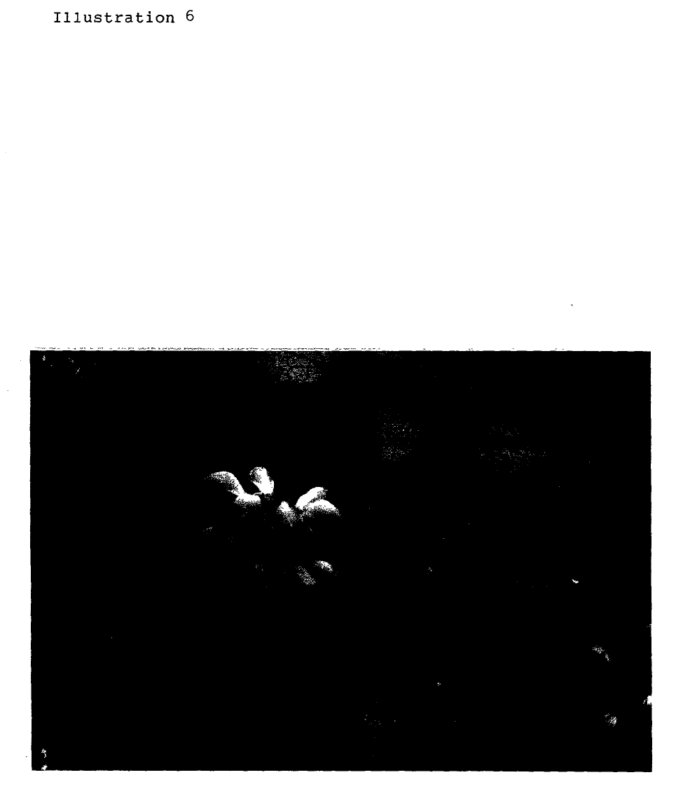

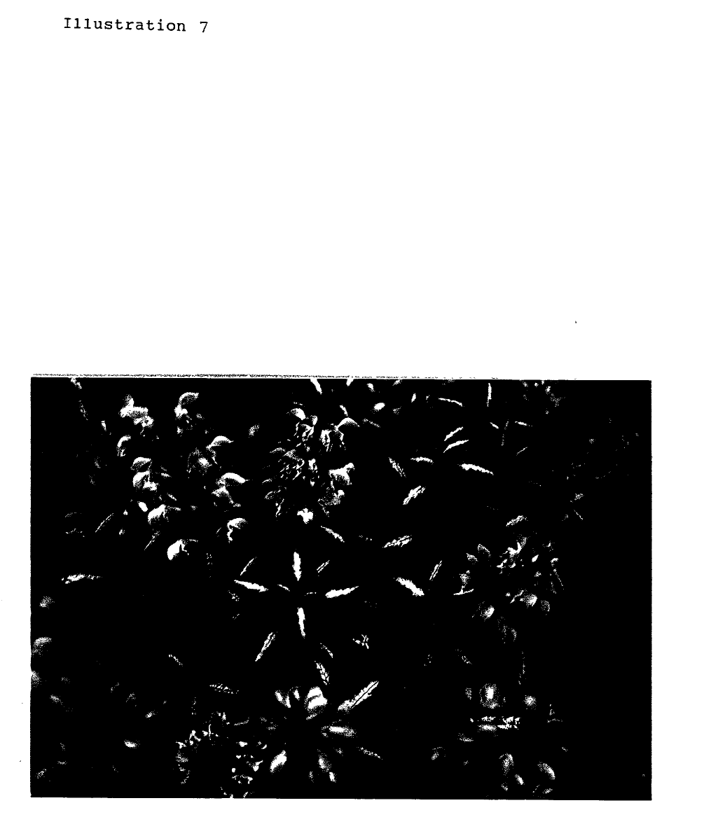

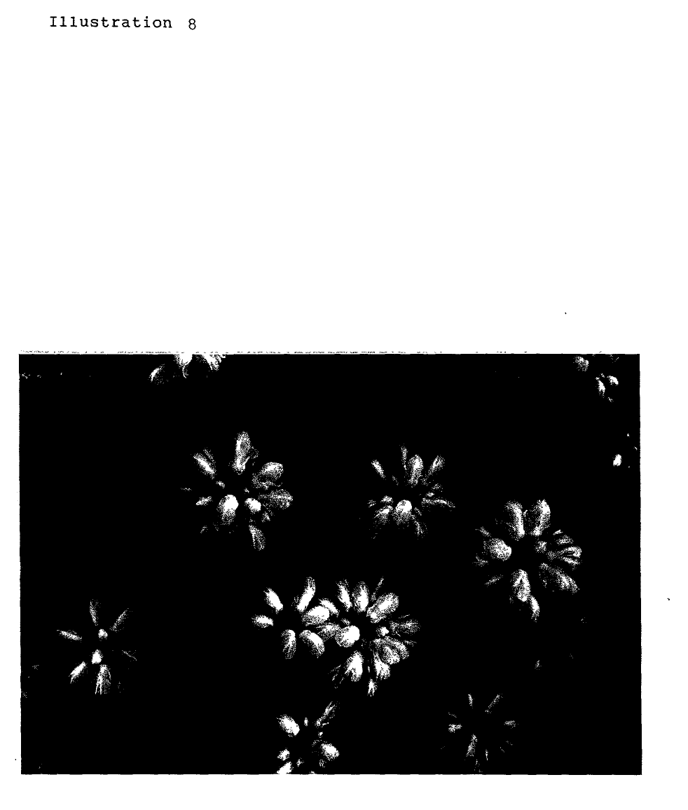

### CROSS REFERENCE

This application is similar to the application previously submitted by Mike Bovio (in which a patent was granted) for his plant, Lamium maculatum ‘Orchid Frost’.

### FEDERALLY SPONSORED RESEARCH

This application does not relate to any federally sponsored research.

### BACKGROUND OF INVENTION

‘Purple Dragon’ originated as a branch mutation from Lamium maculatum ‘White Nancy’, which has similar foliage but white flowers. The first successful asexual reproduction of ‘Purple Dragon’ was carried out by its discoverer, John vanSanden. This propagation took place under controlled conditions at Spring Lake, Mich., on Sep. 1, 2000.

The sport was excised, the bottom leaves removed, treated with IBA, and rooted under intermittent mist over the period of two weeks. Five cuttings rooted and were transplanted into nursery containers for evaluation.

### SUMMARY OF INVENTION

After six months in the nursery containers, the plants were transplanted to growing beds for further evaluation. After observing them in the landscape, the previously noted unique features continued to be observed. The plant has since spread and matured and has flowered repeatedly, each time displaying rich purple flowers.

### DESCRIPTION OF THE VARIOUS VIEWS OF THE DRAWINGS

The accompanying photographic illustrations show typical foliage of the cultivar and the closest known comparison cultivars, with color being as accurate as possible with renditions of this type. The photographs were taken at 1 P.M. on Mar. 10, 2001 under partly sunny conditions, in Spring Lake, Mich. The film used was Kodak Gold ISO 100 exposed-for 1/250 second at F4 using no filters.

Illustration 1 is a perspective view of ‘Purple Dragon’.

Illustration 2 is a perspective view of L. maculatum ‘Beacon Silver’,

Illustration 3 is a perspective view of L. maculatum ‘Chequers’,

Illustration 4 is a perspective view of L. maculatum ‘Orchid Frost’,

Illustration 5 is a perspective view of L. maculatum ‘Pink Pewter’,

Illustration 6 is a perspective of view of L. maculatum ‘Red Nancy’,

Illustration 7 is a combined photo of L. maculatum ‘Shell Pink’, and

Illustration 8 is a perspective view of L. maculatum ‘White Nancy’.

### DETAILED BOTANICAL DESCRIPTION

‘Purple Dragon’ has not been observed under all possible environmental conditions, and the phenotype may vary significantly with variations in environment, such as temperature, light intensity and day length. The following observations, measurements, and comparisons describe this plant as grown in Spring Lake, Mich. when container grown (outdoors without cover) in conditions that approximate those generally used in commercial practice.

The following traits have been repeatedly observed and are determined to be the basic characteristics of ‘Purple Dragon’, which in combination distinguish this dead nettle as a new and distinct cultivar. The color codes correspond to The Royal Horticultural Society Color Chart.

1. Stems of this cultivar arise at acute angles to the growing medium. As they elongate their weight causes them to droop over and sprawl horizontally—usually contacting the growing medium and rooting in to the soil. Typically they reach 12 or more inches in length and often produce branches at their nodes.

2. Typical stems are herbaceous and are uniformly colored light green 139-D in youth maturing to grayed-purple 184-A. The stems are square in cross section and 3/16 inch across.

3. Internodes range in length from ¾ to 2½ inches. The nodes are 3/16 inch across and often sprout roots after a few weeks of contact with the growing medium. Thereafter, the plants generate a pair of new shoots from the buds on the distal end of the nodes.

4. Evergreen foliage is simple and arranged in opposite pairs. In youth, the foliage is ovate in outline, crenate edged, and colored either light green 145-A (about 25 percent of the new leaves are colored this way), or bicolored with the same coloration as the mature leaves (see # 5 below).

5. Mature leaves are cordate, crenate or crentate-dentate margined. Ranging from 2 inches long by 1¼ to 1½ inches wide, they are attached by ¾-1¼ inch long light green petioles 135-D. Hair covered, they are somewhat velvety to the touch. Each is flat, with obtuse apices.

6. The midribs are not prominent and are only slightly recessed into the blade. Colored medium green 143-A, they give rise to a network of secondary and terciary veins of the same color.

7. Adult leaves are bicolored. The center is colored gray-green 192-C, and the leaf edge green 141-B. The leaf edge surrounds the entire leaf blade and is typically ⅛ inch wide.

8. Bisexual flowers are arranged in verticillasters (dense whorls) and are borne primarily during late spring and early summer then throughout the summer and fall months. Each whorl may produce up to 30 flowers, but not all at the same time.

9. Individual flowers are bilabiate (typical of the mint family). The flowers are typically composed of five medium green 143-A, linear, divergent, lobed calyxes. Each corolla is approximately 8/10 inch long by 7/16 inch wide, two-lipped, upper lip hooded, lower lip 3-lobed.

10. The funnel-like base of each corolla is colored white 155-D. The upper lip is purple violet 81-C on the inside and purple violet 80-A on its outside. The midrib of the upper lip is white 155-B, ½ inch long, 1/32 inch wide, and divided distally into two thin forks. The lateral lower lips are colored purple 78-A on all sides. The central lower lip is colored purple 77-A on both both sides.

11. The fruit, grouped in fours, is unnoticable with casual observation. Each is a tiny ⅛ inch long, 1/16 inch wide, green 135-D nutlet.

Other Registered and Related Cultivars of Spotted Dead Nettle are:

‘Beacon Silver’, with flowers somewhat smaller ¾ inch long by 7/16 inch wide, colored red-purple 68-A on the outside of the upper lip of the corolla, and leaves with similar variegation and coloring (to ‘Purple Dragon’).

‘Chequers’, with rose-pink flowers and substantially different leaves which are smaller (¾ inch long by ¾ inch wide) and centered gray-green 193-D with a broad ⅜ inch wide dark green 137-A margin.

‘Orchid Frost’, with violet colored flowers, the upper lip violet 84-C won the inside and violet 84-A on its outside.

‘Pink Pewter’ which displays light pink flowers 73-D and smaller foliage, to 1⅛ inch long and 1 inch wide, colored silvery 192-B with thin 1/16 inch wide green 136-B margin.

‘Pink Pewter’, has ¾ inch long flowers that are colored red-purple 73-D. Its leaves are typically 1¼ inch long by ⅝ inch wide. Colored gray-green 192-B in their center, they are surrounded with an irregular ⅕ inch wide margin of dark green 137-D.

‘Red Nancy’, has ¾ inch long flowers that are colored violet 84-B. Its leaves are typically 1 inch long by ¾ inch wide. Colored gray-green 192-B in their center, they are surrounded with a thin, 1/10 inch wide margin of dark green 137-D.

‘Shell Pink’, with ¾ inch long flowers that are colored pink 62-C, and leaves smaller, to 1 inch long and 1 inch wide, that are centered gray-green 192-A with a broad ½ inch wide margin of dark green 137-B.

‘White Nancy’, with white 155-D flowers measuring ¾ inch long by 7/16 inch wide, and leaves with similar variegation, size, and coloring (to ‘Orchid Frost’).

1. The present invention comprises a new and distinct cultivar of Lamium maculatum, hereafter referred to by the cultivar name ‘Purple Dragon’. ‘Purple Dragon’ is a new and distinct variety of spotted dead nettle, an herbaceous perennial from Europe, ‘Purple Dragon’ is unique and outstanding for its purple flowers, and in addition for its colorful silvery centered, green-edged foliage.
